(19) United States
(12) Patent Application Publication
(10) Pub. No.: US 2013/0323295 A1
(43) Pub. Date: Dec. 5, 2013

(54) MONOMER SYSTEMS WITH DISPERSED SILICONE-BASED ENGINEERED PARTICLES

(71) Applicant: Johnson & Johnson Vision Care, Inc., (US)

(72) Inventors: Charles W. Scales, St. Augustine, FL (US); Eric R. George, St. Augustine, FL (US); Christopher D. Anderson, Wescosville, PA (US); Robert D. Gleim, New Hope, PA (US); Brent Matthew Healy, Jacksonville Beach, FL (US)

(73) Assignee: Johnson & Johnson Vision Care, Inc., Jacksonville, FL (US)

(21) Appl. No.: 13/682,677

(22) Filed: Nov. 20, 2012

Related U.S. Application Data
(60) Provisional application No. 61/568,308, filed on Dec. 8, 2011.

(51) Int. Cl. A61K 9/00; A61K 38/13

(52) U.S. Cl. CPC A61K 9/0051; A61K 38/13
USPC 424/429; 514/20.5

(57) ABSTRACT

Provided are compositions containing engineered particles, and methods of making such engineered particles. Polymeric articles, such as contact lenses, prepared from such compositions are also provided. Such engineered particles are dispersible in hydrophilic systems such as monomer systems for preparation of contact lenses. Each of the engineered particles comprises a hydrophobic core and a hydrophilic shell. The hydrophobic core comprises a silicone-based polymer that can have multiple cross-links and/or polymer-polymer entanglement, and the hydrophilic shell is formed from a reactive stabilizer. A residue of the reactive stabilizer or a hydrophilic segment of the reactive stabilizer can form the shell. The particles have an average particle size of less than about 500 nm.

### RELATED APPLICATIONS

This application claims priority to U.S. Provisional Patent Application No. 61/568,308, filed on Dec. 8, 2011 entitled MONOMER SYSTEMS WITH DISPERSED SILICONE-BASED ENGINEERED PARTICLES, the contents of which are incorporated by reference.

### TECHNICAL FIELD

The invention relates to polymeric articles, such as contact lenses, comprising engineered particles and processes for forming such articles. The engineered particles, which generally comprise a hydrophobic core and a hydrophilic shell, are dispersible in hydrophilic systems such as monomer systems for preparation of contact lenses.

### BACKGROUND

Polymeric materials are desirable for a number of applications, including medical devices. One such application is contact lenses.

Gas permeable soft contact lenses (“GPSCL”) have been made from conventional and silicone hydrogels. Conventional hydrogels have been prepared from monomeric mixtures predominantly containing hydrophilic monomers, such as 2-hydroxyethyl methacrylate (“HEMA”), N-vinyl pyrrolidone (“NVP”), and vinyl alcohol.

Silicone hydrogels (SiH's) are used as materials in GPSCLs. Silicone hydrogels have typically been prepared by polymerizing mixtures containing at least one silicone-containing monomer or reactive macromer and at least one hydrophilic monomer. This class of lens material is desirable because it reduces the corneal edema and hyper-vasculature associated with conventional hydrogel lenses. Such materials, however, can be difficult to produce because the silicone components and the hydrophilic components are incompatible.

There is a need, therefore, to provide silicone-containing monomers or reactive macromers that are compatible with hydrophilic systems, such as monomer systems for contact lenses.

### SUMMARY

Provided are compositions containing engineered particles that have a hydrophobic core and a hydrophilic shell, and methods of making such engineered particles. Polymeric articles, such as contact lenses, prepared from such compositions are also provided. Such engineered particles are dispersible in hydrophilic systems such as monomer systems for preparation of contact lenses

In a first aspect, contact lenses are formed from a composition comprising a plurality of engineered particles having an average particle size of less than about 500 nm dispersed in a monomer system, each of the engineered particles comprising a hydrophobic core and a hydrophilic shell. The hydrophobic core comprises a silicone-based polymer comprising multiple cross-links and the hydrophilic shell is formed from a reactive stabilizer, wherein a residue of the reactive stabilizer covalently bonds to the silicone-based polymer to form the particles. The contact lens has a center thickness in the range of about 50 to about 180 micron and a haze that is less than 100% as compared to a CSI lens.

Another aspect provides compositions that comprise a plurality of engineered particles having an average particle size of less than about 500 nm dispersed in a monomer system, each of the engineered particles comprising a hydrophobic core and a hydrophilic shell, wherein the core comprises a silicone-based RAFT-polymer, which is a reaction product of at least one silicone reactive monomer and a hydrophobic segment of a reactive stabilizer comprising an amphiphilic macro-RAFT agent, and the shell comprises hydrophilic segments of said amphiphilic macro-RAFT agent.

A further aspect is a method of preparing a plurality of engineered particles for dispersion in a monomer system, the method comprising: providing a solution comprising a reactive stabilizer; adding one or more siloxy monomers or macromers and a cross-linker to the solution to form a mixture; emulsifying the mixture to form a mini-emulsion; polymerizing the mini-emulsion to form a polymeric dispersion that comprises a plurality of engineered particles, each of which comprises a hydrophobic polymeric core and a hydrophilic shell, wherein the hydrophilic shell is formed from the reactive stabilizer. A residue of the reactive stabilizer covalently bonds with the siloxy-containing component(s) to form the silicone-based polymer which forms the particles. A second residue of the reactive stabilizer or one or more hydrophilic segments of the reactive stabilizer can form the shell. When the mixture comprises the cross-linker, the core is cross-linked.

In one or more embodiments, the concentration of the engineered particles is increased in the polymeric dispersion by removing solution solvent to form a concentrated dispersion, which is subsequently added into the monomer system.

### BRIEF DESCRIPTION OF THE DRAWINGS

FIG. 1 provides chemical structures of an exemplary set of compositions, including Formula I, which is a reactive stabilizer, Formula II, which is a cross-linker, and Formula III, which is a siloxy macromer;

FIG. 2 provides chemical structures of an exemplary set of compositions, including Formula IV, which is a reactive stabilizer, and Formula V, which is a cross-linker in general form, and Formula VI, which is a siloxy macromer in general form;

FIG. 3 provides the chemical synthesis of an exemplary reactive stabilizer, Formula VII;

FIG. 4 provides another synthesis for formation of engineered particles including Formula VIII, which is a reactive stabilizer, and Formula IX, which shows the reaction of a siloxy macromer and a cross-linker;

FIG. 5 is a three-dimensional surface plot of Particle Rₕ plotted as a function of PEG MW and SiMAA2 DM % by weight;

FIG. 6 is a three-dimensional surface plot of Particle R_{g} plotted as a function of PEG MW and SiMAA₂ DM % by weight; and

FIG. 7 is a three-dimensional surface plot of Particle p plotted as a function of PEG MW and SiMAA2 DM % by weight.

FIG. 8 is an optical micrograph of 50:50 weight ratio mixture of Example 5 dispersion and HEMA.

FIG. 9 is an optical micrograph of 50:50 weight ratio mixture of comparative/prior art Example 17 dispersion and HEMA.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Provided are compositions and contact lenses made from such compositions that comprise silicone-containing engineered particles, such as those that provide oxygen permeability to the contact lenses. The formation of these silicone-containing engineered particles may be accomplished through a variety of techniques, including micro-emulsion or mini-emulsion polymerization and variants/combinations of the same. Disclosed herein are two non-limiting, but preferred routes to forming useful silicone-containing engineered particles via mini-emulsion polymerization for use in contact lenses. It has been found that the use of reactive stabilizers, such as water-soluble free radical initiators, having functional end groups and emulsifying capabilities, can result in the formation of engineered particles of desired properties. Such desired engineered particle properties could include, but are not restricted to, particles that are comprised of a core/shell structure, where the core is composed of a cross-linked, hydrophobic polymers and copolymers (e.g. poly monomethacryloxypropyl terminated mono-n-butyl terminated polydimethylsiloxane (poly(mPDMS)) and copolymers thereof) and the shell is composed of a hydrophilic and potentially biocompatible polymers and copolymers (e.g. polyethylene glycol (PEG), poly(N,N-dimethylacrylamide) (PDMA), polyvinylpyrrolidone (PVP), etc., and copolymers thereof), which is a residue of the reactive stabilizer. Core/shell structured particles can also be of a form where the core is composed of a hydrophobic polymer that is a reaction product of at least one silicone reactive monomer and a hydrophobic segment of a reactive stabilizer that comprises an amphiphilic macro-RAFT agent, and the shell comprises one or more hydrophilic segments of said amphiphilic macro-RAFT agent. Providing the hydrophobic core with a hydrophilic shell yields two desired properties inherent to the particles described in this invention: 1) the ability to disperse the otherwise hydrophobic particles into a polar medium, such as water, polar organic solvents, or polar reactive monomer mixes and 2) the ability to sequester the hydrophobic core away from contact with human tissue, thereby “passivating” the hydrophobic material.

As demonstrated herein, formation of silicone-based engineered particles is accomplished by, but not restricted to mini-emulsion polymerization. In forming these silicone-based engineered particles via mini-emulsion polymerization, molecular weight of the reactive stabilizer is a contributor to obtaining stable and spherical particles. In addition to molecular weight, the reactive stabilizer can also be of an acceptable ability to reduce surface tension between the continuous and discrete phases of the polymerization solution while remaining its initiation activity in order to form a stable mini-emulsion and subsequent engineered particle. Preparation of engineered particles of desired sizes and surface properties allows for dispersion of generally hydrophobic polymers into hydrophilic systems. Such engineered particles may also be suitable for delivering therapeutic agents. The engineered particles can have an average particle size of less than about 500 nm. In one or more embodiments, the average particle size is in the range of about 1 to 300, about 5 to 250 nm (or even about 100 to 225 nm).

Reference to “mini-emulsion” or “miniemulsion” means emulsions which are typically free of non-reactive, small molecule surfactants. In a miniemulsion, the stabilizer (typically polymeric or oligomeric) is incorporated into the particle covalently or through physical entanglement or a combination thereof.

Reference to “stable” means that the silicon-based engineered particles do not settle or aggregate in a solution, at room temperature, as evidenced by coagulum which is visible under an optical microscope for a defined period of at least about 2 months, about 6 months and in some embodiments about 1 year.

Reference to “dispersed” means particles are substantially uniformly distributed in a monomer system such that there is minimal aggregation of the particles. In one or more embodiments, the particles are dispersed in a monomer system in an amount to maximize the presence of particles in the monomer system without saturating the system and rendering it too thick to flow. In one embodiment, the particle loading is up to about 70%. Other detailed embodiments provide that the particle loading in the monomer system is in the range of about 30 to about 70% by weight (or about 35 to about 65%, or even about 39 to about 62%). The particles desirably deliver to the monomer systems elemental Si in the range of about 4 to about 10% by weight (or about 5 to about 9%, or even about 6 to about 8%)

Reference to “reactive stabilizer” means a compound that is capable of reducing the interfacial surface tension between the continuous phase and discrete phase of two immiscible liquids and is capable of reaction with the discrete phase components under the selected polymerization conditions. It has been surprisingly found that for the engineered particle systems of the present invention, the reactive stabilizers contain functional groups provided by a polymeric or oligomeric polymerization initiator, such as a PEG-functional diazo-macroinitiator, or a polymerization mediating agent, such as, but not limited to an amphiphilic macro-RAFT agent, that imparts hydrophilic stability to the resulting polymer during a polymerization reaction of one or more materials that are hydrophobic so that the resulting polymer can be dispersed in water, a polar organic solvent, or a polar monomer system. The term “RAFT” means reversible addition-fragmentation chain transfer. The hydrophilic portion of the above-mentioned amphiphilic reactive stabilizers, whether based on a macro-RAFT agent or a diazo-macro-initiator, can be comprised of oligomeric materials. In the case of the amphiphilic reactive stabilizers comprising diazo-macro-initiators, at elevated temperatures the diazo groups thermally degrade into N₂, leaving two polymeric or macromeric free radicals and liberating N₂ gas. The remaining hydrophilic free radicals are referred to herein as “the “residue” of the reactive stabilizer and are thus left to initiate polymerization at the interface of the particle and/or covalently bond to the particle core. For particles prepared via RAFT mini-emulsion polymerization, an amphiphilic macro-RAFT agent is employed to disperse/stabilize hydrophobic silicone monomer(s) in an aqueous solution. When the amphiphilic macro-RAFT agent contains its reactive thiocarbonylthio group at the hydrophobic terminus of the polymer, the reactive thiocarbonylthio-group can participate in and control the polymerization of the dispersed hydrophobic silicone monomer droplet, thus forming a polymeric particle that is stabilized/dispersed by an outer-shell of covalently anchored hydrophilic segments derived from those of the hydrophilic portion of the original amphiphilic macro-RAFT agent. Such oligomeric species could include, but are not limited to polyalkylene glycol, polyamides and polyhydroxy alkyl (meth)acrylate polymers and copolymers. Specific examples include, but are not limited to polyethylene glycol (PEG, as mentioned above), poly(N,N-dimethylacrylamide) (PDMA) polyvinylpyrrolidone (PVP), poly(2-hydroxypropylmethacrylamide) (PHEMA), poly(N-2-hydroxypropylmethacrylamide) (PHPMA) poly(N,N-dimethylacrylamide-co-3-acrylamidopropanoic acid) (poly(DMA-co-ACA1.0), poly(N,N-dimethylacrylamide-co-4-acrylamidobutanoic acid) (poly(DMA-co-ACA1.5), poly(N,N-dimethylacrylamide-co-5-acrylamidopentanoic acid) (poly(DMA-co-ACA2.0), and combinations thereof and the like.

Reference to “shell” means a hydrophilic layer on the core that provides at least partial and at most complete surrounding and/or encapsulation of the core. The hydrophilic nature of the shell results in stability of individual particles not only during their formation in an aqueous solution but also upon dispersion of the particles into a monomer system. The shell is covalently bonded to the polymer of the core of the particle. In one or more embodiments, the shell itself may be cross-linked. Reference to “core” means a polymer that is encapsulated and partitioned from the continuous phase by the shell. The core comprises multiple “cross-links,” between polymer chains which means it is held together by multiple covalent bonds. The core can also comprise polymer-polymer entanglement. Both cross-links and polymer-polymer entanglement provide mechanical integrity to the core. Also a cross-linker can bring additional functionality within the core. For example, in one embodiment, the cross-linker can be a di-functional polydimethylsiloxane.

Reference to “monomer system” or “reactive monomer mix (RMM)” or “reaction mixture” means a mixture of components, including, reactive components, diluent (if used), initiators, cross-linkers and additives, which when subjected to polymer forming conditions form a polymeric hydrogel material. Typically, such mixtures include at least one monomer suitable for polymerization into a flexible plastic material, such as contact lenses. Reactive components are the components in the reaction mixture, which upon polymerization, become a permanent part of the polymer, either via chemical bonding, entrapment or entanglement within the polymer matrix. Monomer systems can include hydrophilic monomers. Classes of monomers that can be desirable for monomer systems include acrylates, methacrylates, acrylamides, methacrylamides, styrenes, n-vinyl monomers, and o-vinyl monomers. An exemplary methacrylate includes 2-hydroxyethyl methacrylate (HEMA) and an exemplary methyacrylamide includes N,N dimethyacrylamide (DMA). N-vinyl monomers can include, but are not limited to, N-vinyl pyrrolidone and N-vinyl acetamide. An exemplary O-vinyl monomer is O-vinyl acetate.

Reference to “therapeutic agent” means a drug or other material or mixture of the same that provides benefit to a recipient. Exemplary therapeutic agents include, but are not limited to immunosuppressant drugs, anti-microbial agents, antifungal agents, vitamins, anti-inflammatory agents, anti-VEGF (vascular epithelial growth factor) agents, macular pigment supplements, antibiotics, intraocular pressure reducing agents, and the like, and combinations thereof. In one or more embodiments, a rate of therapeutic agent release (controlled drug release) is controlled by the chemistry of the core and shell of the particle and the matrix material. Permeability is defined as the product of diffusion rate of the permeant (the therapeutic agent) and the solubility of the permeant within a given medium (the combination of the core/shell particle and its given matrix). The rate of therapeutic agent release will be directly related to the permeability as defined here. For example, when the chemistry and size of the therapeutic agent changes relative to the core and shell of the particle and resulting matrix material a change of release rate (exiting the contact lens) will occur.

As used herein “biocompatibility” and “biocompatible” means that the material in question does not cause any substantial negative response when in contact with the desired biological system. For example when the oxygen permeable particles are incorporated into contact lenses some undesirable negative responses could include stinging, inflammation, undesirable levels of protein and lipid uptake, ocular cell damage and other immunological responses. Preferred embodiments of the silicone engineered particles of this invention would not evoke such undesirable negative responses in the body.

A “hydrogel” polymer is a polymer capable of absorbing or imbibing at least about 20 weight % water, in some embodiments at least about 30 weight % water and in other embodiments at least about 40 weight % water and yet in other embodiments at least about 60 weight % water.

Reference to “substantially surfactant-free” means that a conventional latex surfactant, which is a non-reactive, small molecule is in one embodiment not added to the composition. It is possible, however, that small amounts of surfactant (less than about 10%, less than about 1% and in some embodiments less than about 0.5%) may be employed for a plurality of reasons, e.g. addition of surface active agents to a mini-emulsion to promote smaller particle sizes.

As used herein “clarity” means substantially free from visible haze. Clear lenses have a haze value of less than about 150%, more preferably less than about 100% as compared to a CSI Thin Lens®.

In a detailed embodiment, the reactive stabilizer is present upon preparation of the particles in a ratio of about 3:1 by weight of a mixture of the siloxy macromer and cross-linker with the reactive stabilizer. Other contemplated weight ratios include about 10:1 (or about 5:1, or even about 0.5:1).

The shell of the particle can comprise about 50% or more up to about 100% by weight of the residue of the reactive stabilizer. Specifically, the shell can comprise about 50% (or about 60%, or about 70%, or about 80%, or about 90%, or about 95%, or about 99%, or even about 100%) by weight of the residue.

The reactive stabilizers have molecular weights to form particles of desired sizes and stabilities. In one or more examples, the molecular weight is in the range of about 1000 to about 9000 g/mol (or about 2000 to about 4000 g/mol or about 5000 to about 8000 g/mol).

The core of the particles is generally a silicone-based hydrophobic polymer, which can comprise multiple cross-links and/or entangled polymers. The silicone-based hydrophobic polymers are generally formed from one or more siloxy monomers or macromers and one or more cross-linkers. Siloxy monomers and macromers are generally mono-functional in that one end of the compound is targeted for polymerization. Cross-linkers are generally having at least two functional groups to participate in cross-linking. In one or more embodiments, the cross-linkers can be siloxy-functional.

In a detailed embodiment, the total siloxy-containing components are present with the total cross-linkers upon preparation of the particles in a ratio of about 50:50 by weight, that is, 50:50 wt/wt siloxy-containing component to cross-linker. Other contemplated weight ratios ranges could include about 100:0 to 0:100 (or about 80:20 to 20:80, or even about 60:40 to 40:60). The hydrophobic core can comprise siloxy-containing component in the range of about 0.1 to about 50% by weight (or about 20 to 50% or even about 45-50%).

The siloxy-containing components include, but are not limited to, polydialkyl siloxanes, such as mPDMS (monomethacryloxypropyl terminated mono-n-butyl terminated polydimethylsiloxane) or OHmPDMS (mono-(3-methacryloxy-2-hydroxypropyloxy)propyl terminated, mono-butyl terminated polydimethylsiloxane)), SiMAA₂ (Methyl-bis(trimethylsilyloxy)-silyl-propylglycerol-methacrylate), polydialkylsiloxane acrylamides, in some embodiments polydimethylsiloxane acrylamides, such as SA1, SA2, and those listed in US 20110237766 or combinations thereof.

Other siloxy-containing components include those that contains at least one [—Si—O—Si] group, in a monomer, macromer, or prepolymer. In one embodiment, the Si and attached O are present in the siloxy-containing component in an amount greater than 20 weight percent, and in another embodiment greater than 30 weight percent of the total molecular weight of the siloxy-containing component. Useful siloxy-containing components comprise polymerizable functional groups such as acrylate, methacrylate, acrylamide, methacrylamide, N-vinyl lactam, N-vinylamide, and styryl functional groups. Examples of silicone-containing components which are useful in this invention may be found in U.S. Pat. Nos. 3,808,178; 4,120,570; 4,136,250; 4,153,641; 4,740,533; 5,034,461 and 5,070,215, and EP80539. All of the patents cited herein are hereby incorporated in their entireties by reference. These references disclose many examples of olefinic silicone-containing components.

Suitable siloxy-containing components include compounds of Formula I:

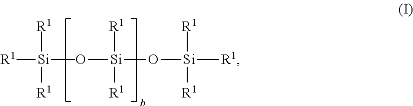

R¹ is independently selected from monovalent reactive groups, monovalent alkyl groups, or monovalent aryl groups, any of the foregoing which may further comprise functionality selected from hydroxy, amino, oxa, carboxy, alkyl carboxy, alkoxy, amido, carbamate, carbonate, halogen or combinations thereof; and monovalent siloxane chains comprising 1-100 Si—O repeat units which may further comprise functionality selected from alkyl, hydroxy, amino, oxa, carboxy, alkyl carboxy, alkoxy, amido, carbamate, halogen or combinations thereof;

where b=0 to 25, where it is understood that when b is other than 0, b is a distribution having a mode equal to a stated value;

wherein at least one R¹ comprises a monovalent reactive group, and in some embodiments only one or two R¹ comprise a monovalent reactive group.

As used herein “monovalent reactive groups” are groups that can undergo free radical and/or cationic polymerization. Non-limiting examples of free radical reactive groups include (meth)acrylates, styryls, vinyls, vinyl ethers, C1-6alkyl(meth)acrylates, (meth)acrylamides, C₁₋₆alkylmeth)acrylamides, N-vinyllactams, N-vinylamides, C₂₋₁₂alkenyls, C₂₋₁₂alkenylphenyls, C₂₋₁₂alkenylnaphthyls, C₂₋₆alkenylphenylC₁₋₆alkyls, O-vinylcarbamates and O-vinylcarbonates. Non-limiting examples of cationic reactive groups include vinyl ethers or epoxide groups and mixtures thereof. In one embodiment the free radical reactive groups comprises (meth)acrylate, acryloxy, (meth)acrylamide, and mixtures thereof.

Suitable monovalent alkyl and aryl groups include unsubstituted monovalent C₁ to C₁₆ alkyl groups, C₆-C₁₄ aryl groups, such as substituted and unsubstituted methyl, ethyl, propyl, butyl, 2-hydroxypropyl, propoxypropyl, polyethyleneoxypropyl, combinations thereof and the like.

In one embodiment b is zero, one R¹ is a monovalent reactive group, and at least 3 R¹ are selected from monovalent alkyl groups having one to 16 carbon atoms, and in another embodiment from monovalent alkyl groups having one to 6 carbon atoms. Non-limiting examples of silicone components of this embodiment include 2-methyl-,2-hydroxy-3-[3-[1,3,3,3-tetramethyl-1-[(trimethylsilyl)oxy]disiloxanyl]propoxy]propyl ester (“SiGMA”),

2-hydroxy-3-methacryloxypropyloxypropyl-tris(trimethylsiloxy)silane,

3-methacryloxypropyltris(trimethylsiloxy)silane (“TRIS”),

3-methacryloxypropylbis(trimethylsiloxy)methylsilane and

3-methacryloxypropylpentamethyl disiloxane.

In another embodiment, b is 2 to 20, 3 to 15 or in some embodiments 3 to 10; at least one terminal R¹ comprises a monovalent reactive group and the remaining R¹ are selected from monovalent alkyl groups having 1 to 16 carbon atoms, and in another embodiment from monovalent alkyl groups having 1 to 6 carbon atoms. In yet another embodiment, b is 3 to 15, one terminal R¹ comprises a monovalent reactive group, the other terminal R¹ comprises a monovalent alkyl group having 1 to 6 carbon atoms and the remaining R¹ comprise monovalent alkyl group having 1 to 3 carbon atoms. Non-limiting examples of silicone components of this embodiment include (mono-(2-hydroxy-3-methacryloxypropyl)-propyl ether terminated polydimethylsiloxane (400-1000 MW)) (“OH-mPDMS”), monomethacryloxypropyl terminated mono-n-butyl terminated polydimethylsiloxanes (800-1000 MW), (“mPDMS”).

In another embodiment, b is 2 to 20, 3 to 15 or in some embodiments 3 to 10; at least two R¹ comprises a monovalent reactive group and the remaining R¹ are selected from monovalent alkyl groups having 1 to 16 carbon atoms, and in another embodiment from monovalent alkyl groups having 1 to 6 carbon atoms. In yet another embodiment, b is 3 to 15, one terminal R¹ comprises a monovalent reactive group, the other terminal R¹ comprises a monovalent alkyl group having 1 to 6 carbon atoms and the remaining R¹ comprise monovalent alkyl group having 1 to 3 carbon atoms. Non-limiting examples of silicone components of this embodiment include monomethacryloxypropyl terminated mono-n-butyl terminated polydimethylsiloxane dimethacrylate (mPDMS DM:).

In another embodiment, one to four R¹ comprises a vinyl carbonate or carbamate of Formula II:

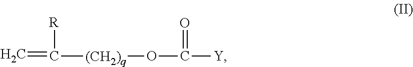

wherein: Y denotes O—, S— or NH—;

R denotes, hydrogen or methyl; d is 1, 2, 3 or 4; and q is 0 or 1.

The silicone-containing vinyl carbonate or vinyl carbamate monomers specifically include: 1,3-bis[4-(vinyloxycarbonyloxy)but-1-yl]tetramethyl-disiloxane; 3-(vinyloxycarbonylthio)propyl-[tris(trimethylsiloxy)silane]; 3-[tris(trimethylsiloxy)silyl]propyl allyl carbamate; 3-[tris(trimethylsiloxy)silyl]propyl vinyl carbamate; trimethylsilylethyl vinyl carbonate; trimethylsilylmethyl vinyl carbonate, and

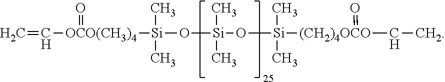

Siloxy-containing components generally have molecular weights less than about 5000 daltons.

In one embodiment, the silicone content of the particles may be further enriched by addition of a silicone oil to the mini-emulsion mixture prior to sonication and curing. Such systems could be useful in applications where very high silicone contents are desirable.

Incorporation of a cross-linking agent during the curing process helps to stabilize the engineered particles. Suitable cross-linkers are compounds with two or more polymerizable functional groups. Selection of cross-linking agents depends on the functionality of the siloxy-containing component employed in particle formation. Any suitable cross-linker with two or more functional groups can aid in interparticle bonding and strengthening of the polymer. In one embodiment, the particles enhance the oxygen permeability of the polymer systems to which they are added. In this embodiment, preferred cross-linkers comprise silicone in order to impart as much oxygen permeability as possible to the particles. Examples of silicone cross-linking agents are well known to those skilled in the art and include, but are not limited to SiMAA2 DM (Methyl-bis(trimethylsilyloxy)-silyl-propylglycerol-dimethacrylate), tetra-alkoxy silanes and poly-functional vinyl, allyl, or silyl-hydride moieties with appropriate hydrosilylating metal catalysts. Additional cross-linkers include, but are not limited to: mPDMS DM (monomethacryloxypropyl terminated mono-n-butyl terminated polydimethylsiloxane dimethacrylate); difunctional (cross-linking) silicone monomers such as bis(3-methacryloxypropyl)polydimethylsiloxane, bis(4-methacryloxybutyl)polydimethylsiloxane, 1,3-bis(3-methacryloxypropyl)tetrakis(trimethylsiloxy)disiloxane, and others as disclosed in U.S. Pat. Nos. 4,260,725; 5,034,461; 5420324 and 5,760,100. While the core of the particles may be cross-linked with either a hydrophobic or hydrophilic cross-linker, the former embodiment is preferred to minimize migration of the cross-linker from the cores of the stabilized mini-emulsion monomer droplets to the aqueous phase, where undesirable polymerization and subsequent mini-emulsion destabilization might take place. Appropriate selection of hydrophobic cross-linkers over those that are hydrophilic for the purpose of cross-linking the silicone cores should be apparent to those skilled in the art. When forming the final hydrogel material, i.e. the lens material, the cross-linker may be hydrophilic or hydrophobic and in some embodiments of the present invention mixtures of hydrophilic and hydrophobic cross-linkers have been found to provide silicone hydrogels with improved optical clarity (reduced haze compared to a CSI Thin Lens). Examples of suitable hydrophilic cross-linkers include compounds having two or more polymerizable functional groups, as well as hydrophilic functional groups such as polyether, amide or hydroxyl groups. Specific examples include TEGDMA (tetraethyleneglycol dimethacrylate), TrEGDMA (triethyleneglycol dimethacrylate), ethyleneglycol dimethacrylate (EGDMA), ethylenediamine dimethyacrylamide, glycerol dimethacrylate and combinations thereof and the like. Examples of suitable hydrophobic cross-linkers include multifunctional hydroxyl-functionalized silicone containing monomer, multifunctional polyether-polydimethylsiloxane block copolymers, combinations thereof and the like. Specific hydrophobic cross-linkers include SiMAA2 dimethacrylate, OHmPDMS dimethacrylate, mPDMS dimethacrylate, acryloxypropyl terminated polydimethylsiloxane (n=10 or 20) (acPDMS), hydroxylacrylate functionalized siloxane macromer, butanediol dimethacrylate, divinyl benzene, 1,3-bis(3-methacryloxypropyl)-tetrakis(trimethylsiloxy)disiloxane and mixtures thereof.

In one embodiment, preferred agents for preparing cross-linked engineered silicone particles include, but are not limited to, mPDMS DM, acPDMS, SiMAA₂ DM, OHmPDMS DM and combinations thereof and the like. Preferred cross-linkers to be used to prepare the final lens hydrogel material include TEGDMA, EGDMA, acPDMS and combinations thereof and the like. The cores of engineered silicone particles may be cross-linked with as much as 60% wt/wt of cross-linker in the mini-emulsion total monomer feed. In the final hydrogel material, the amount of hydrophilic cross-linker used is generally about 0 to about 2 weight % and preferably from about 0.5 to about 2 weight % and the amount of hydrophobic cross-linker is about 0 to about 5 weight %, which can alternatively be referred to in mol % of about 0.01 to about 0.2 mmole/gm reactive components, preferably about 0.02 to about 0.1 and more preferably 0.03 to about 0.6 mmole/gm.

Increasing the level of cross-linker in the final lens hydrogel material has been found to reduce the amount of haze. However, as cross-linker concentration increases above about 0.15 mmole/gm reactive components modulus increases above generally desired levels (greater than about 90 psi). Thus, in the present invention the cross-linker composition and amount is selected to provide a cross-linker concentration in the reaction mixture of between about 0.01 and about 0.1 mmoles/gm cross-linker.

One or more embodiments provide that the composition is substantially surfactant-free. Under micro-emulsion conditions such as those disclosed in Examples 12-15 of US2010/0249273, conventional latex surfactants (small molecule, nonreactive surfactants) are typically used to maintain stability of the emulsions. With mini-emulsion conditions and the formation of shells around cores of the particles, the use of such conventional latex surfactants is not typically necessary to maintain stability, but can be desirable in small amounts to maintain, for example, particle size.

While particles free of conventional latex surfactants are preferred, some embodiments of this invention may include conventional latex surfactant present in amounts of up to 10 wt %. Conventional latex surfactants include small molecule surfactants, polymeric surfactants, amphiphilic copolymers, combinations thereof and the like. Examples of Conventional latex surfactants include alkyl ethyoxylates (Brij Surfactants), alkyl/aryl sulfonates and sulfates (e.g. dodecylbenzenesulfonate or sodium dodecylsulfate), PEG-120 Methyl Glucose Dioleate (DOE 120, commercially from Lubrizol), PVP, polyvinyl alcohol/polyvinyl acetate copolymers, amphiphilic statistical or block copolymers such as silicone/PVP block copolymers, polyalkylmethacrylate/hydrophilic block copolymers, organoalkoxysilanes such as 3-aminopropyltriethoxysilane (APS), methyl-triethoxysilane (MTS), phenyl-trimethoxysilane (PTS), vinyl-triethoxysilane (VTS), and 3-glycidoxypropyltrimethoxysilane (GPS), silicone macromers having molecular weights greater than about 10,000 and comprising groups which increase viscosity, such as hydrogen bonding groups, such as but not limited to hydroxyl groups and urethane groups and mixtures thereof.

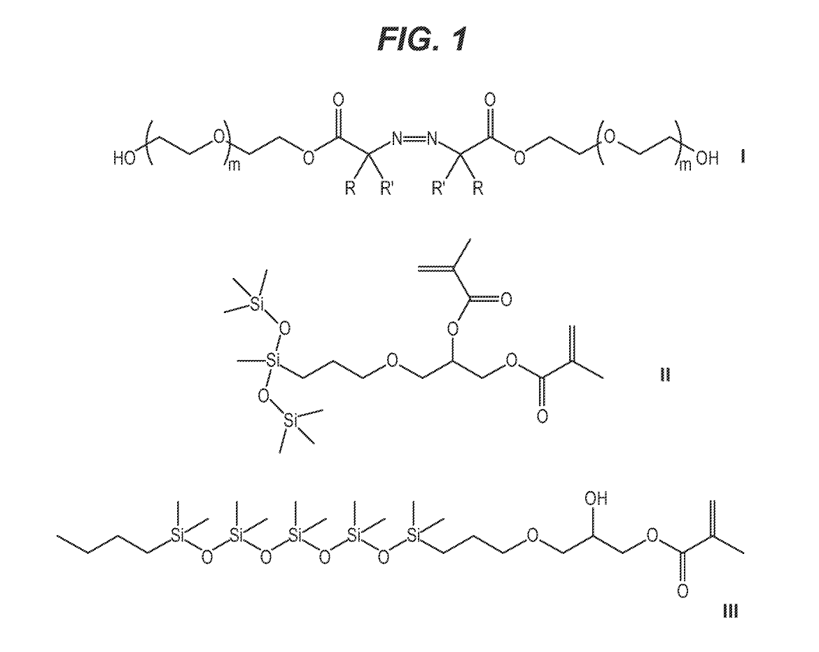

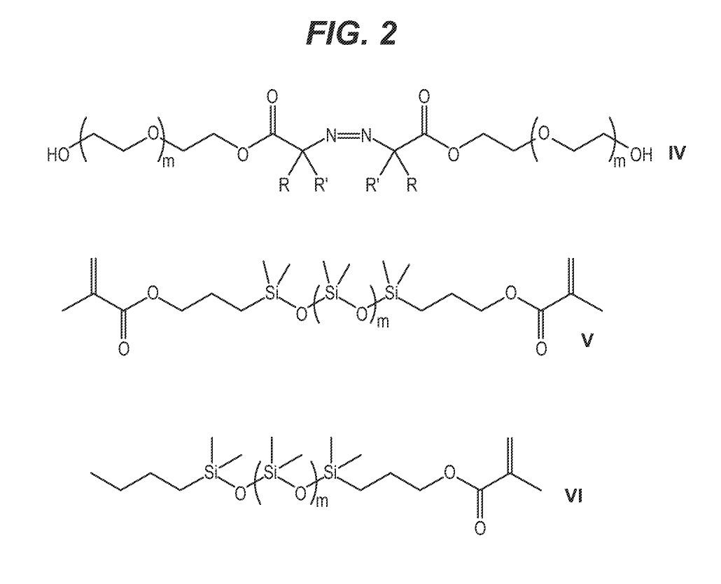

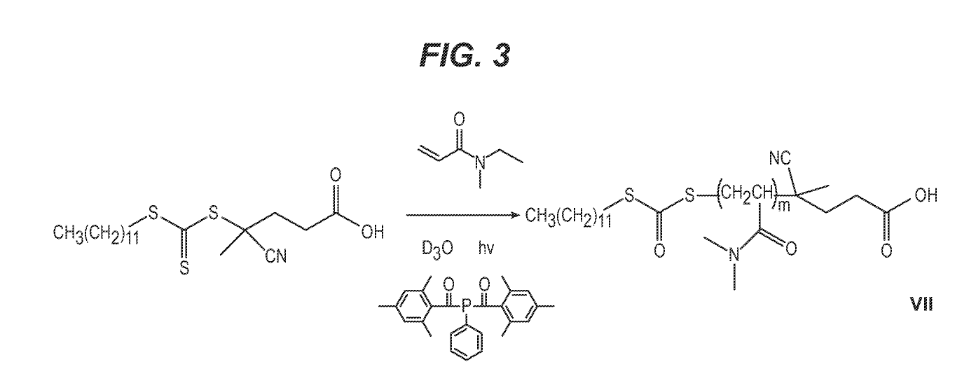

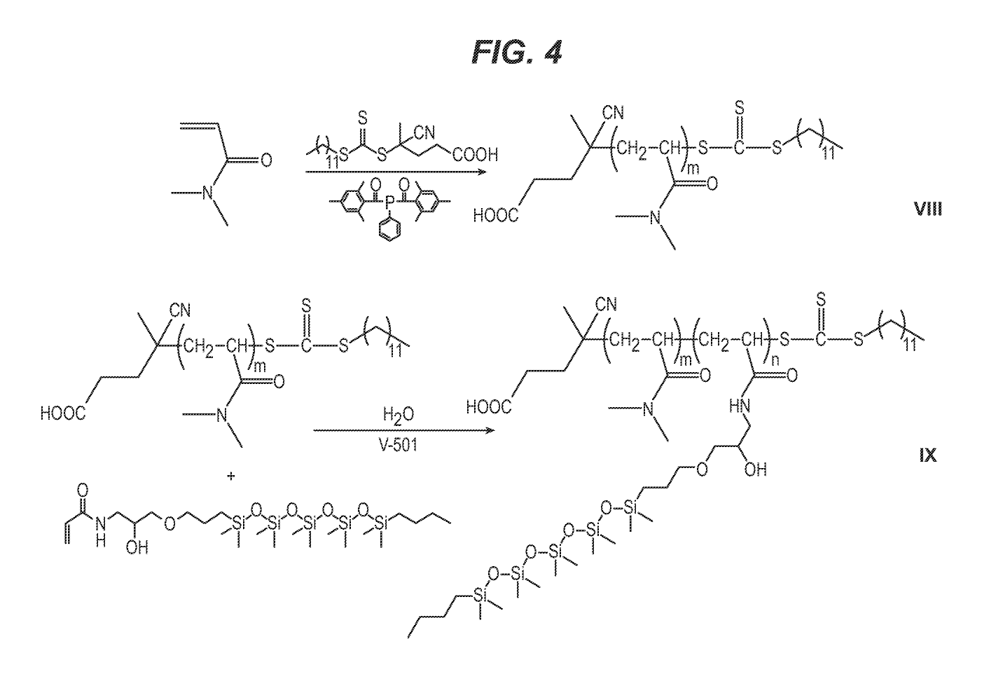

Turning to the figures, FIG. 1 shows chemical formulas for components of a composition of an embodiment. A reactive stabilizer that is polyethylene glycol diazo macroinitiator (Formula I, which is polyethylene glycol diazo macroinitiator) is first provided in a solvent such as water to form a solution. A siloxy macromer (Formula III, which is OHmPDMS) and a cross-linker (Formula II, which is SiMAA₂) are added and formation of engineered silicone particles proceeds as discussed in detail in the examples. FIG. 2 provides another exemplary set of compositions that can be used together, where Formula IV, polyethylene glycol diazo macroinitiator, is the reactive stabilizer Formula V is the general formula for a suitable Si-containing dimethacrylate cross-linker, and Formula VI is the general formula for a suitable siloxy macromer. FIG. 3 shows the chemical synthesis for a reactive stabilizer (Formula VII) according to one embodiment. That is, as desired, the reactive stabilizer itself can be synthesized for subsequent use in making the engineered particles. FIG. 4 shows another synthesis for formation of engineered particles using the reactive stabilizer of FIG. 3, where Formula VIII depicts Formula VII in a slightly different arrangement and Formula IX shows the synthesis of another embodiment.

In general terms, preparation of the particles can be done at temperatures and pressures as desired and consistent with conventional manufacturing processes. The initial particle preparation can take place at room temperature (typically in the range of about 19-25° C.) without much need to go higher and ambient pressure. It is preferred that the water-soluble reactive stabilizer be added as the first component to an aqueous mixture.

The addition in any order of the siloxy-containing component and the cross-linker usually occurs after the addition of the reactive stabilizer. These materials can be added dropwise or all at once as needed. Emulsifying the mixture is done under conditions conducive to forming mini-emulsions, which means agitating or even sonicating under conditions of sufficient time and energy to obtain particles of desired size. Depending on the energy of mixing, duration for emulsifying can range from about 10 seconds to about 10 minutes (or even about 10 to about 30 seconds or even about 1 to about 5 minutes) Temperature can range widely (want to stay below 100° C. to avoid boiling the water) and is usually done under ambient conditions of temperature and pressure.

Polymerization of the mini-emulsion can occur thermally or be photoinitiated. For polymerization at elevated temperatures, the range is about 60-80° C., or even about 70-75° C. for up to 24 hours (specifically 12-18 hours) to a point where substantially all of the monomer is consumed. For photoinitiation, the system can be exposed to UV or other suitable light source until substantially all of the monomer is consumed.

The finished emulsion, or polymeric dispersion, can be concentrated by removing the solvent, usually water, used in preparation of the reactive stabilizer solution to a desired % solids by weight, in the range of about 50-75%, such as 50%, 55%, 60%, 65%, 70% or even 75%. The solvent can be removed by any known means. The concentrated dispersion can then be added to the monomer system. Alternatively, the un-concentrated dispersion can be added to the monomer system and the final stable monomer/particle dispersion can be concentrated by removing solvent.

The compositions of the present invention have a balance of properties that makes them particularly useful. In one embodiment, the compositions having engineered silicone particles of a particular size are used to make lenses, and particularly contact lenses, where such properties include elevated oxygen transmissibility (Dk), wettability, improved biocompatibility, and optical clarity. Thus, in one embodiment, the biomedical devices are contact lenses made from a composition having an average particle size of less than about 200 nm dispersed in a monomer system, and the lenses have a center thickness (CT) in the range of about 50 to about 180 micron and less than 100% haze compared to a CSI lens.

In specific embodiments, the engineered particles are oxygen permeable particles that are selected so that they do not substantially degrade the optical properties of the polymer, including color and clarity. This may be accomplished by controlling the particle size, refractive index, chemical properties of the oxygen permeable particles or any combination of the foregoing. The oxygen permeable particles have a refractive index of within about 20% hydrated polymer matrix and in some embodiments within about 10% of the refractive index of the hydrated polymer matrix. Other embodiments may employ oxygen permeable particles with a refractive index within about 1% of the hydrated polymer matrix and in other embodiments still, less than 0.5%. In one embodiment, the oxygen permeable particles have an average particle size between about 200 and about 1000 nm and a refractive index within about 10% of the refractive index of the hydrated polymer matrix. Oxygen permeable particles with a particle size of less than 200 nm, may have refractive indices which are within about 20% of the refractive index of said hydrated polymer matrix. In one embodiment, where the polymer is a hydrogel suitable for making contact lenses, the refractive index of the oxygen permeable particle is between about 1.37 and about 1.47. In one embodiment the refractive index of the hydrogel polymer is between about 1.39 and about 1.43 and the oxygen permeable particles have a refractive index within the ranges specified above. The contact lens can have an oxygen permeability in the range of about 10 to about 20 barrer more than a comparative contact lenses without the particles

Haze Measurement

Haze is measured by placing a hydrated test lens in borate buffered saline in a clear 20×40×10 mm glass cell at ambient temperature above a flat black background, illuminating from below with a fiber optic lamp (Dolan-Jenner PL-900 fiber optic light with 0.5″ diameter light guide set at a power setting of 4-5.4) at an angle 66° normal to the lens cell, and capturing an image of the lens from above, normal to the lens cell with a video camera (DVC 1300C:19130 RGB camera with Navitar TV Zoom 7000 zoom lens) placed 14 min above the lens platform. The value of the background scatter (BS) is measured using a saline filled glass cell which is captured using EPIX XCAP V 2.2 software. The subtracted scattered light image is quantitatively analyzed, by integrating over the central 10 mm of the lens, and then comparing to a −1.00 diopter CSI Thin Lens®, which is arbitrarily set at a “CSI haze value” of 100, with no lens set as a haze value of 0. Five lenses are analyzed and the results are averaged to generate a haze value as a percentage of the standard CSI lens.

Alternatively, instead of a −1.00 diopter CSI Thin Lenses®, a series of aqueous dispersions of stock latex spheres (commercially available as 0.49 μm Polystyene Latex Spheres—Certified Nanosphere Size Standards from Ted Pella, Inc., Product Number 610-30) can be used as standards. A series of calibration samples were prepared in deionized water. Each solution of varying concentration was placed in a cuvette (2 mm path length) and the solution haze was measured using the above method.

| | Concentration | |
|---|---|---|
| Solution | (wt % × 10⁻⁴) | Mean GS |
| 1 | 10.0 | 533 |
| 2 | 6.9 | 439 |
| 3 | 5.0 | 379 |
| 4 | 4.0 | 229 |
| 5 | 2.0 | 172 |
| 6 | 0.7 | 138 |

Mean GS = mean gray scale

A corrective factor was derived by dividing the slope of the plot of Mean GS against the concentration (47.1) by the slope of an experimentally obtained standard curve, and multiplying this ratio times measured scatter values for lenses to obtain GS values.

“CSI haze value” may be calculated as follows:

CSI haze value=100×(GS−BS)/(217−BS)

Where GS is gray scale and BS is background scatter.

Water Content

The water content of contact lenses was measured as follows: Three sets of three lenses are allowed to sit in packing solution for 24 hours. Each lens is blotted with damp wipes and weighed. The lenses are dried at 60° C. for four hours at a pressure of 0.4 inches Hg or less. The dried lenses are weighed. The water content is calculated as follows:

$$\% water content = \frac{( wet weight - dry weight )}{wet weight} \times 100$$

The average and standard deviation of the water content are calculated for the samples and are reported.

Oxygen Permeability (Dk)

Oxygen permeability (Dk) for silicone lenses was determined by the polarographic method generally described in ISO 9913-1: 1996(E), but with the following variations. The measurement is conducted at an environment containing 2.1% oxygen. This environment is created by equipping the test chamber with nitrogen and air inputs set at the appropriate ratio, for example 1800 ml/min of nitrogen and 200 ml/min of air. The t/Dk is calculated using the adjusted oxygen concentration. Borate buffered saline was used. The dark current was measured by using a pure humidified nitrogen environment instead of applying MMA lenses. The lenses were not blotted before measuring. Four lenses were stacked instead of using lenses of varied thickness. A curved sensor was used in place of a flat sensor. The resulting Dk value is reported in barrers.

Asymmetric Flow Field Flow Fractionation with Multi-Angle Laser Light Scattering and Quasi-Elastic Light Scattering (AFFF-MALLS-QELS)

The absolute size distributions for particles disclosed herein were determined by AFFF-MALLS-QELS. Generally, AFFF is a fractionation technique known for its ability to fractionate particles of various sizes, including polymers, proteins, and nano-particles that are less than 10 nm in size and larger particles up to a few microns in size. In a typical AFFF separation, the smaller structures elute from the fractionation chamber first and are followed by larger particles. As used in this invention, AFFF is employed in the fractionation of silicone particles into a distribution of sizes which can be analyzed simultaneously with in-line MALLS and QELS detectors to give radius of gyration and radius of hydration data, respectively. The technique is particularly useful in determining the absolute size distributions of particles that have very broad ranges in size. This is because each discrete particle size, within the distribution of sizes for a given sample, can be separated, sized, and quantified during elution, thus yielding a true distribution of particle sizes.

The AFFF-MALLS-QELS setup employed a Wyatt Eclipse™3+ AFFF system, Wyatt DAWN Treos™ MALLS detector, Wyatt QELS detector (multiple tau correlation design), and a Wyatt OptilabT-rEX refractive index detector (Wyatt Technology Corporation, Santa Barbara, Calif., USA). The chromatography conditions for all AFFF-MALLS-QELS experiments included using a 20 mM phosphate buffer (pH 7.4) with 200 ppm NaN₃ (to prevent microbial growth) as an eluent and employed the use of a 10 kD Nadir membrane with a 350 μm spacer in the fractionation chamber. The volumetric channel flow rate was maintained at 1 mL/min while the initial cross-flow was set at 3 mL/min. Data was analyzed using the ASTRA V software package (Wyatt Technology Corporation, Santa Barbara, Calif., USA). A gradient cross-flow program was used to fractionate each sample and elute it into the attached MALLS and QELS detectors for size analysis.

Prior to analysis, the MALLS 90 degree detector was calibrated with toluene and the other detectors were normalized to the 90 degree detector with bovine serum albumin. All particle samples were diluted with 0.2 μm filtered phosphate eluent to a final concentration of 10 mg/mL.

The Examples below further describe this invention, but do not limit the invention. They are meant only to suggest a method of practicing the invention. Those knowledgeable in the field of contact lenses as well as other specialties may find other methods of practicing the invention. However, those methods are deemed to be within the scope of this invention.

Some of the materials employed in the Examples are identified as follows:

EGDMA: ethyleneglycol dimethacrylate;

HEMA: 2-hydroxyethyl methacrylate (99% purity);

MAA: methacrylic acid (99% purity);

BzMA: benzyl methacrylate;

OHmPDMS: mono-(3-methacryloxy-2-hydroxypropyloxy)propyl terminated, mono-butyl terminated polydimethylsiloxane), (612 molecular weight), DSM Polymer Technology Group;

OH PDMS dimethacrylate

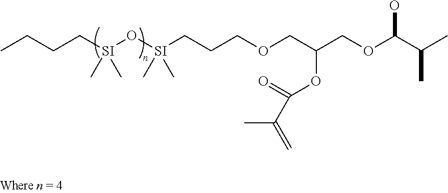

SiMAA2 (Methyl-bis(trimethylsilyloxy)-silyl-propylglycerol-methacrylate);

SiMAA2 DM:

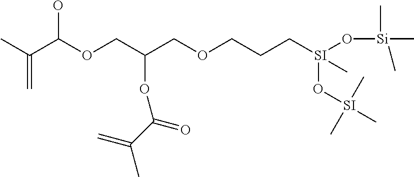

DMA: N,N-dimethylacrylamide;

PDMA: polydimethylacrylamide;

mPDMS-900: monomethacryloxypropyl terminated mono-n-butyl terminated polydimethylsiloxane (900 molecular weight), Gelest

mPDMS DM:

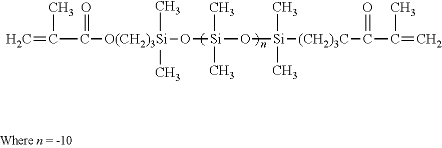

SA1: N-(3-(3-(9-butyl-1,1,3,3,5,5,7,7,9,9-decamethylpentasiloxanyl)propoxy)-2-hydroxypropyl)acrylamide) as shown in the following formula:

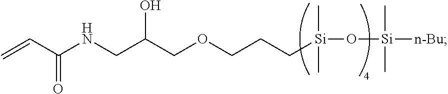

SA2: as shown in the following formula:

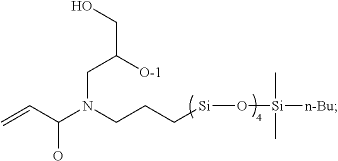

V-501 diazo-initiator ((Z)-4,4′-(diazene-1,2-diyl(bis(4-cyanopentanoic acid);

VPE-0201: 2000 g/mole PEGylated diazo-initiator (PEG functional diazo-initiator where the PEG has a molecular weight of 2000 g/mole);

VPE-0401: 4000 g/mole PEGylated diazo-initiator (PEG functional diazo-initiator where the PEG has a molecular weight of 4000 g/mole);

VPE-0601: 6000 g/mole PEGylated diazo-initiator (PEG functional diazo-initiator where the PEG has a molecular weight of 6000 g/mole);

DTTC-PA: 4-cyano-4-[(dodecylsulfanylthiocarbonyl)sulfanyl]pentanoic acid;

CGI-819: a photo-initiator, Irgacure 819 (Bis(2,4,6-trimethylbenzoyl)-phenylphosphineoxide);

CGI-1700: a photo-initiator, Irgacure 1700 (75/25% (wt) blend of 2-hydroxy-2-methyl-1-phenyl-propan-1-one and bis(2,6-dimethoxybenzoyl)-2,4,4-trimethylpentyl phosphine oxide) (CAS #189750-87-6).

### EXAMPLES

The following non-limiting examples shall serve to illustrate various embodiments of the present invention.

### Example 1

### Copolymerization of Siloxane Methacrylates at Varying Ratios Using Polyethylene Glycol Azo Macroinitiator (MW=4,000 g/mol)

Several silicone monomer dispersions with polyethylene glycol azo macroinitiator, VPE-0401 were prepared. The weight ratio of the siloxy macromer OHmPDMS and the cross-linker SiMAA₂ DM was varied according to Table 1 below.

TABLE 1
Compositions of SiMAA₂ DM and OHmPDMS mixtures employed in
preparing VPE-0401-stabilized silicone particles via mini-emulsion
polymerization

| Experiment | Example 1A | Example 1B | Example 1C |
|---|---|---|---|
| SiMAA₂ DM | 80 wt % | 45 wt % | 20 wt % |
| OHmPDMS | 20 wt % | 55 wt % | 80 wt % |

Generally, polyethylene glycol diazo-macroinitiator (VPE-0401, Wako USA, MW 4,000 g/mol) (3 grams) was dissolved in DI water (9 grams) and then the appropriate siloxane methacrylate comonomer mixture (3 grams) at the desired SiMAA₂ DM/OHmPDMS composition shown in Table 1 was added. The monomer was emulsified by pipette mixing, followed by sonication for a total of 30 seconds (in 3×10 sec intervals) at a power level of 7 using the Fisher Scientific Model 550 Sonic Dismembrator. The resulting opaque white emulsions were then polymerized overnight at 70° C. at 60 rpm in a rotary oven.

The finished latexes were viscous white fluids with no visible coagulum present. Example 1A appeared to be the most opaque, whereas Example 1C appeared to be the most translucent. Under the microscope, a few small aggregates were present, but the latexes were generally well-dispersed. The latexes were freely soluble in DI water and in HEMA, resulting in translucent viscous fluids. The appearance of the latexes under the optical microscope did not change after dilution.

To more fully characterize the PEG-stabilized silicone microparticles, the absolute size distributions of the dispersions were measured via AFFF-MALLS-QELS. Sizing results for Examples 1A, 1B, and 1C are shown in Table 2.

TABLE 2
Radius of Hydration (Rₕ), Radius of Gyration (R_{g}), and Shape Factor (ρ)
data for SiMAA₂ DM/OHmPDMS engineered particles prepared with
4000 MW VPE-0401

| | | SiMAA₂ DM | Number | Number | |
|---|---|---|---|---|---|
| Particle | PEG | OHmPDMS | Average Rh | Average | Shape |
| Name | MW | (wt ratio) | (nm) | Rg (nm) | Factor ρ |
| Example 1A | 4000 | 80/20 | 47.0 | 67.0 | 1.43 |
| Example 1B | 4000 | 45/55 | 41.0 | 47.5 | 1.16 |
| Example 1C | 4000 | 20/80 | 37.5 | 39.0 | 1.04 |

### Example 2

### Copolymerization of Siloxane Methacrylates at Varying Ratios Using Polyethylene Glycol Azo Macroinitiator (MW=6,000 g/mol)

Several silicone monomer dispersions with polyethylene glycol azo macroinitiator were prepared in the same manner as Example 1, except that the PEG azo initiator molecular weight was increased to 6,000 g/mol (VPE-0601, Wako USA, MW 6,000 g/mol). The siloxy macromer to cross-linker weight ratios (Table 3), and the methods of preparation, were the same as those employed in Example 1.

TABLE 3
Compositions of SiMAA₂ DM and OHmPDMS mixtures employed in
preparing VPE-0601-stabilized silicone particles via mini-emulsion
polymerization

| Experiment | Example 2A | Example 2B | Example 2C |
|---|---|---|---|
| SiMAA₂ DM | 80 wt % | 45 wt % | 20 wt % |
| OHmPDMS | 20 wt % | 55 wt % | 80 wt % |

The resulting latexes were white fluids with no visible coagulum. They were generally more viscous and opaque than those of Example 1. The latexes were easily redispersible in HEMA, and showed no signs of aggregation under the optical microscope. Samples of each dispersion were analyzed via AFFF-MALLS-QELS. Sizing results for Examples 2A, 2B, and 2C are shown in Table 4.

TABLE 4
Radius of Hydration (Rₕ), Radius of Gyration (R_{g}), and Shape Factor (ρ)
data for SiMAA₂ DM/OHmPDMS engineered particles prepared with
6000 MW VPE-0601

| | | SiMAA₂ DM | Number | Number | |
|---|---|---|---|---|---|
| Particle | PEG | OHmPDMS | Average Rh | Average | Shape |
| Name | MW | (wt ratio) | (nm) | Rg (nm) | Factor ρ |
| Example 2A | 6000 | 80/20 | 64.0 | 119.0 | 1.86 |
| Example 2B | 6000 | 45/55 | 54.0 | 82.5 | 1.53 |
| Example 2C | 6000 | 20/80 | 51.5 | 75.0 | 1.46 |

Based on Examples 1 and 2, the particle size was directly proportional to the molecular weight of the PEG azo macroinitiator and to the SiMAA₂ DM:OHmPDMS ratio.

### Example 3

### Copolymerization of Siloxane Methacrylates at Varying Ratios Using Polyethylene Glycol Azo Macroinitiator (MW=2,000 g/mol)

Several silicone monomer dispersions with polyethylene glycol azo macroinitiator were prepared in the same manner as Example 1, except that the PEG azo initiator molecular weight was decreased to 2,000 g/mol (VPE-0201, Wako USA, MW 2,000 g/mol). The siloxy macromer to cross-linker weight ratios (Table 5), and the methods of preparation, were the same as in Example 1.

TABLE 5
Compositions of SiMAA₂ DM and OHmPDMS mixtures employed in
preparing VPE-0201-stabilized silicone particles via mini-emulsion
polymerization

| Experiment | Example 3A | Example 3B | Example 3C |
|---|---|---|---|
| SiMAA₂ DM | 80 wt % | 45 wt % | 20 wt % |
| OHmPDMS | 20 wt % | 55 wt % | 80 wt % |

The resulting latexes were translucent white fluids with no visible coagulum. They were generally less viscous and more translucent than Examples 1 and 2, suggesting a smaller particle size. The latexes were easily redispersible in HEMA. Examples 3A and 3B showed no signs of aggregation under the optical microscope. Example 3C, however, began to precipitate in HEMA, as evidenced by very small particulates on the microscopic level. Samples of each dispersion were analyzed via AFFF-MALLS-QELS. Sizing results for Examples 3A, 3B, and 3C are shown in Table 6.

TABLE 6
Radius of Hydration (Rₕ), Radius of Gyration (R_{g}), and Shape Factor (ρ)
data for SiMAA₂ DM/OHmPDMS engineered particles prepared with
2000 MW VPE-0201

| | | SiMAA₂ DM | Number | Number | |
|---|---|---|---|---|---|
| Particle | PEG | OHmPDMS | Average Rh | Average | Shape |
| Name | MW | (wt ratio) | (nm) | Rg (nm) | Factor ρ |
| Example 3A | 2000 | 80/20 | 62.0 | 62.0 | 1.00 |
| Example 3B | 2000 | 45/55 | 42.0 | 41.5 | 0.99 |
| Example 3C | 2000 | 20/80 | 38.5 | 38.5 | 1.00 |

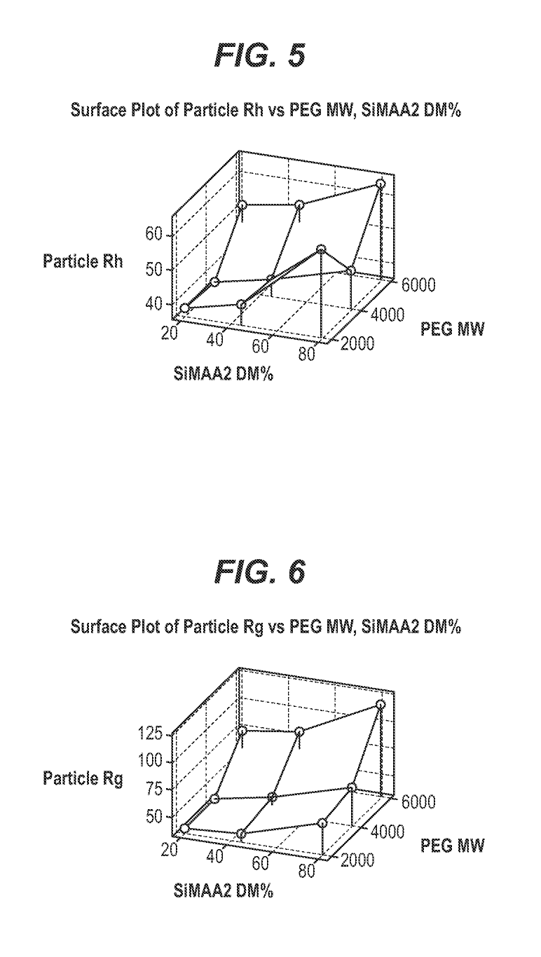

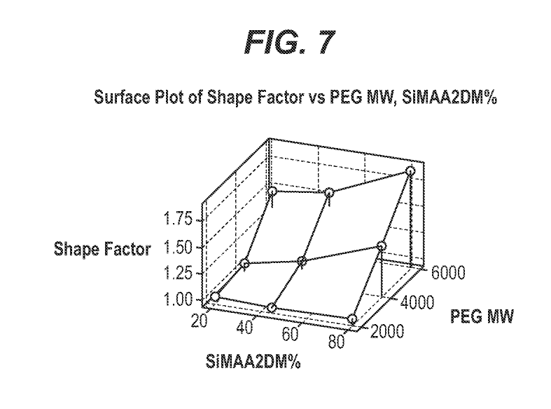

Based on Examples 1, 2, and 3, the particle size was generally directly proportional to the molecular weight of the PEG diazo-macroinitiator and to the SiMAA₂ DM:OHmPDMS weight ratio. Without intending to be bound by theory, it is believed that there are three factors that greatly impact particle size and stability, including 1) the length/size of the hydrophilic stabilizing PEG oligomer, 2) the number of reactive sites available for interfacial reaction with the mini-emulsion monomer droplet, and 3) the amount of silicone monomer:cross-linking silicone monomer. If one considers a finite surface area of a stabilized mini-emulsion monomer droplet stabilized by PEG, it becomes evident that there is a point at which the benefit of longer stabilizing lengths of PEG is diminished by the fact that larger PEG oligomers bring fewer reactive groups to the interface to react with silicone monomer than smaller PEG oligomers. Conversely, if the length of the PEG stabilizer is too short, it becomes more difficult to provide ample hydrophilic/steric stabilization to the hydrophobic droplet/particle. This can result in particle stability issues seen in some of the data provided in Example 3C. The data in Tables 2, 4, and 6 are combined in Table 7 to illustrate the size dependencies. FIGS. 5, 6, and 7 graphically illustrate the data in Table 7 with three-dimensional surface plots, where Rₕ, R_{g}, and ρ are respectively plotted as functions of PEG MW and SiMAA₂ DM % by weight. Generally, as the shape-factor ρ approaches 1, the particles become more spherical and the size is minimized.

TABLE 7
Combined Radius of Hydration (Rₕ), Radius of Gyration (R_{g}), and
Shape Factor (ρ) data for SiMAA₂ DM/OHmPDMS engineered
particles prepared with 2000, 4000, and 6000 MW macroinitiators,
i.e. VPE-201, VPE-401, and VPE-601, respectively.

| | | SiMAA₂ DM | Number | Number | |
|---|---|---|---|---|---|
| Particle | PEG | OHmPDMS | Average | Average | Shape |
| Name | MW | (wt ratio) | Rₕ (nm) | R_{g} (nm) | Factor ρ |
| Example 3A | 2000 | 80/20 | 62.0 | 62.0 | 1.00 |
| Example 3B | 2000 | 45/55 | 42.0 | 41.5 | 0.99 |
| Example 3C | 2000 | 20/80 | 38.5 | 38.5 | 1.00 |
| Example 1A | 4000 | 80/20 | 47.0 | 67.0 | 1.43 |
| Example 1B | 4000 | 45/55 | 41.0 | 47.5 | 1.16 |
| Example 1C | 4000 | 20/80 | 37.5 | 39.0 | 1.04 |
| Example 2A | 6000 | 80/20 | 64.0 | 119.0 | 1.86 |
| Example 2B | 6000 | 45/55 | 54.0 | 82.5 | 1.53 |
| Example 2C | 6000 | 20/80 | 51.5 | 75.0 | 1.46 |

### Example 4

### Enrichment of OHmPDMS and SiMAA₂ DM in Silicone Particles Prepared Via Mini-Emulsion Polymerization with Polyethylene Glycol Azo Macroinitiator (MW=4,000 g/mol)

In an effort to increase the silicone content of the engineered particles, mini-emulsions with enriched levels of SiMAA₂ DM and OHmPDMS were prepared and polymerized to form stable particles. Three types of particles were prepared with different enrichment levels of a 45:55 blend of SiMAA₂ DM and OHmPDMS. Enrichment of the silicone monomer blend was achieved by targeting three different wt/wt ratios of VPE-0401:silicone monomer blend (e.g. 1:1, 1:2, and 1:3) in the final emulsion. All three mini-emulsion compositions yielded stable particles with very little visible coagulum present. Table 8 shows the compositions that were targeted in each experiment.

TABLE 8
Compositions employed for preparing particles with increased
levels of silicone

| Experiment | Example 4A | Example 4B | Example 4C |
|---|---|---|---|
| SiMAA₂ DM | 22.5 wt % | 30.0 wt % | 33.8 wt % |
| OHmPDMS | 27.5 wt % | 36.7 wt % | 41.3 wt % |
| PEG initiator | 50.0 wt % | 33.3 wt % | 25.0 wt % |
| Blend:PEG | 1:1 | 2:1 | 3:1 |

### Example 5

### Preparation of mPDMS-Based Silicone Particles Via Mini-Emulsion Polymerization with Polyethylene Glycol Azo Macroinitiator (MW=4,000 g/mol)

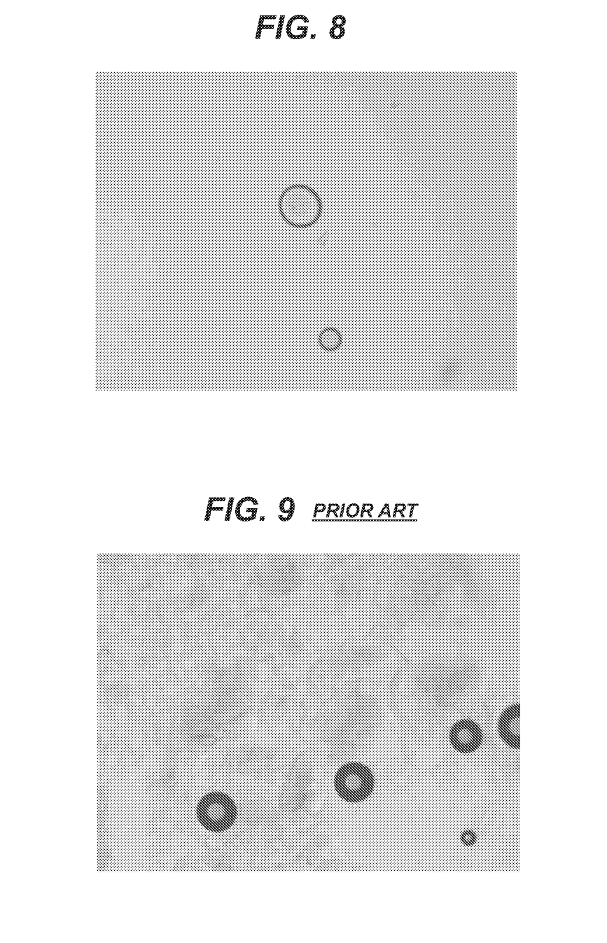

Particles with very high levels of silicone were prepared by substituting the silicone monomers used in Examples 1-4, namely OHmPDMS and SiMAA₂ DM, for mono- and di-methacryloxy-terminal PDMS macromers that are higher in elemental silicone. Particles were composed of a blend of mPDMS-900, mPDMS-DM-1000, mPDMS-5000, and mPDMS-DM-4000. Table 9 below details the specific target compositions that were employed in the preparation of enriched mPDMS-based particles. In all cases, the mini-emulsions were formed with a 1:3 wt/wt ratio of VPE-0401:silicone monomer blend. The resulting latexes were stable and dispersible in 50:50 mixtures with HEMA. In HEMA, the dispersions were translucent liquids. Under optical microscopy, the dispersion in HEMA was substantially free of aggregation, although a few gas bubbles were present, as in FIG. 8.

TABLE 9
Composition of mPDMS and mPDMS-DM blend employed in the
preparation of highly-enriched silicone particles via mini-emulsion
polymerization with VPE-0401

| Silicone Macromer | wt % of Monomer Blend |
|---|---|
| mPDMS-900 | 36.5 |
| mPDMS-DM-1000 | 36.5 |
| mPDMS-5000 | 13.5 |
| mPDMS-DM-4000 | 13.5 |

### Example 6

### Preparation of mPDMS/Perfluorodecyl Methacrylate (PFDMA)-Containing Particles Via Mini-Emulsion Polymerization with Polyethylene Glycol Azo Macroinitiators (MW=2000, and 6,000 g/mol)

Particles containing mixtures of mPDMS and perfluorodecyl methacrylate (PFDMA) were prepared to lower the effective refractive index (RI) of the silicone particles to more closely match the RI of a hydrated contact lens material. Miniemulsion polymerizations were carried out with PFDMA using a slightly modified procedure to that used in preparing particles of Example 5. An aqueous 50:50 by weight blend of VPE-0201:VPE-0601 was prepared. Separately, emulsions of PFDMA/silicone monomer (in varying ratios) were prepared by sonication. The monomers were immediately added to the macroinitiator solutions, mixed, and sonicated to form miniemulsions. The miniemulsions were then polymerized according to standard procedures used in previous examples. The following mini-emulsions with PFDMA and the mPDMS blend from Example 5 were prepared successfully and are listed in Table 10 below. The resulting latexes were stable and dispersible in 50:50 weight ratio mixtures with HEMA. In HEMA, the dispersions were translucent liquids, except Example 6B, which was transparent.

TABLE 10
Mini-emulsion compositions for particles comprising mPDMS and
PFDMA.

| Example | PFDMA | mPDMS |
|---|---|---|
| Number | wt % | Blend wt % |
| Example 6A | 0.0 | 100.0 |
| Example 6B | 22.5 | 77.5 |
| Example 6C | 45.0 | 55.0 |

### Example 7

Monomer compositions were prepared as follows:

TABLE 11A
Compositions for contact lenses.

| | 7A | | 7B | | 7C | | 7D | |
|---|---|---|---|---|---|---|---|---|
| | (wt | 7A | (wt | 7B | (wt | 7C | (wt | 7D |
| Material | %) | (g) | %) | (g) | %) | (g) | %) | (g) |
| Example 5 | 75 | 3.75 | 75 | 5.25 | 75 | 3.75 | 75 | 3.75 |
| CGI-1700 | 0.8 | 0.04 | 0.8 | 0.04 | 0.8 | 0.04 | 0.8 | 0.04 |
| EGDMA | 1.5 | 0.075 | 1.5 | 0.075 | 1.5 | 0.075 | 1.5 | 0.075 |
| MAA | 0 | 0 | 1 | 0.05 | 2 | 0.10 | 3 | 0.15 |
| HEMA | 22.7 | 1.135 | 21.7 | 1.085 | 20.7 | 1.035 | 19.7 | 0.985 |
| Total | 100 | 5 | 100 | 5 | 100 | 5 | 100 | 5 |

For Examples 7A-7D, each monomer composition was diluted by 23% by weight with t-amyl alcohol.

TABLE 11B
Characterization of compositions for contact lenses.

| Material | 7A | 7B | 7C | 7D |
|---|---|---|---|---|
| Water Content (wt %) | 28 | 41 | 50.6 | 58.4 |
| Modulus (PSI) | 144 | 125 | 111 | ND |
| Elongation (%) | 118` | 88 | 56 | ND |
| Toughness | 65 | 28 | 10 | ND |
| Tensile Strength | 112 | 71 | 40 | ND |
| Dk | 38 | 43 | 48 | 53 |
| Haze | 93 | 179 | 365 | 383 |
| RI | 1.4291 | 1.4157 | 1.3996 | 1.3878 |

Contact lenses compositions were prepared in accordance with known procedures. Lenses made from the compositions of Example 7 showed elevated Dk values (as compared to compositions containing less silicone); however, lenses were mechanically weak and most compositions were too low in water content. At higher water contents, the Dk was most elevated, but the lenses were weak and very hazy.

### Example 8.1

Monomer compositions according to Example 7 were prepared in Examples 8.1A-8.1C with the additional ingredient of benzyl methacrylate, which was added to the formula to modulate the RI to match that of the particles at higher water-content values as follows:

TABLE 12A
Compositions for contact lenses.

| | 8.1A | 8.1A | 8.1B | 8.1B | 8.1C | 8.1C |
|---|---|---|---|---|---|---|
| Material | (wt %) | (g) | (wt %) | (g) | (wt %) | (g) |
| Example 5 | 75 | 5.25 | 75 | 5.25 | 75 | 5.25 |
| CGI-1700 | 0.8 | 0.056 | 0.8 | 0.056 | 0.8 | 0.056 |
| EGDMA | 1.5 | 0.105 | 1.5 | 0.105 | 1.5 | 0.105 |
| MAA | 0 | 0 | 1 | 0.07 | 2 | 0.14 |
| HEMA | 17.43 | 1.2201 | 16.43 | 1.1501 | 15.43 | 1.0801 |
| BzMA | 5.27 | 0.3689 | 5.27 | 0.3689 | 5.27 | 0.3689 |
| Total | 100 | 7 | 100 | 7 | 100 | 7 |

For Examples 8.1A-8.1C, each monomer composition was diluted by 26% (by weight) with t-amyl alcohol.

TABLE 12B
Characterization of compositions for contact lenses.

| Material | 8.1A | 8.1B | 8.1C |
|---|---|---|---|
| Water Content | 60.6 | 30.2 | 42.2 |
| (wt %) | | | |
| Modulus (PSI) | 704 | 215 | 150 |
| Elongation (%) | 186 | 142 | 97 |
| Toughness | 425 | 126 | 43 |
| Tensile Strength | 423 | 174 | 90 |
| Dk | 25.8 | 48.5 | 38.7 |
| Haze | 275 | 35 | 95 |
| RI | 1.4306 | 1.4266 | 1.4138 |

Contact lenses compositions were prepared in accordance with known procedures. Lenses made from the compositions of Example 8.1 showed elevated Dk values (as compared to lenses containing less silicone); however, lenses were mechanically stronger than those in Example 7. Also, it was easier to match RI at higher water contents than it was for lenses in Example 7.

### Example 8.2

### Fabrication of Contact Lenses

Contact lenses were prepared in accordance with known procedures. The RMMs had the formulations as provided in Table 13. The particle dispersion used in examples 8.2A-8.2H was the same as in Examples 8.1 and contained 60% by weight solids.

TABLE 13A
Compositions for contact lenses.

| | 8.2A | 8.2A | 8.2B | 8.2B | 8.2C | 8.2C | 8.2C | 8.2C |
|---|---|---|---|---|---|---|---|---|
| Material | (wt %) | (g) | (wt %) | (g) | (wt %) | (g) | (wt %) | (g) |
| Example 5 | 75 | 2.25 | 75 | 2.25 | 75 | 2.25 | 75 | 2.25 |
| CGI-1700 | 0.75 | 0.0225 | 0.75 | 0.0225 | 0.75 | 0.0225 | 0.75 | 0.0225 |
| EGDMA | 1 | 0.03 | 1 | 0.03 | 1 | 0.03 | 1 | 0.03 |
| MAA | 2 | 0.06 | 2 | 0.06 | 2 | 0.06 | 2 | 0.06 |
| HEMA | 18.25 | 0.5475 | 17.25 | 0.5175 | 16.25 | 0.4875 | 15.25 | 0.4575 |
| BzMA | 3 | 0.09 | 4 | 0.12 | 5 | 0.15 | 6 | 0.18 |
| Total | 100 | 3 | 100 | 3 | 100 | 3 | 100 | 3 |

TABLE 13B
Compositions for contact lenses.

| | 8.2D | 8.2D | 8.2E | 8.2E | 8.2G | 8.2G | 8.2H | 8.2H |
|---|---|---|---|---|---|---|---|---|
| Material | (wt %) | (g) | (wt %) | (g) | (wt %) | (g) | (wt %) | (g) |
| Example 5 | 75 | 2.25 | 75 | 2.25 | 75 | 2.25 | 75 | 2.25 |
| CGI-1700 | 0.75 | 0.0225 | 0.75 | 0.0225 | 0.75 | 0.0225 | 0.75 | 0.0225 |
| EGDMA | 1 | 0.03 | 1 | 0.03 | 1 | 0.03 | 1 | 0.03 |
| MAA | 2 | 0.06 | 2 | 0.06 | 2 | 0.06 | 2 | 0.06 |
| HEMA | 14.25 | 0.4275 | 13.25 | 0.3975 | 12.25 | 0.3675 | 11.25 | 0.3375 |
| BzMA | 7 | 0.21 | 8 | 0.24 | 9 | 0.27 | 10 | 0.3 |
| Total | 100 | 3 | 100 | 3 | 100 | 3 | 100 | 3 |

For Examples 9A-9H, each monomer composition was diluted by 26% (by weight) with t-amyl alcohol.

TABLE 14
Characterization of contact lenses.

| Metric | 9A | 9B | 9C | 9D | 9E | 9F | 9G | 9H |
|---|---|---|---|---|---|---|---|---|
| Haze (%) | 167 | 153 | 152 | 131 | 78 | 78 | 84 | 66 |
| RI | 1.4082 | 1.4120 | 1.4114 | 1.4136 | 1.4160 | 1.4186 | 1.4189 | 1.4206 |
| Water Content (%) | 49.9 | 47.4 | 46.4 | 44.6 | 44 | 41.8 | 43.8 | 41 |

All lenses were prepared at −1.0 power using Zeonor (Zeon Chemical) front/back curves. Curing was carried-out in an N₂-purged glove box at 50° C. for 10 minutes under a TL03 lamp (400 nm) at an intensity of 3.4 mW/cm². Lenses were demolded and released in a deionized water-bath at 90° C. prior to being stored in Borate Buffered Saline Solution in individual crimp-sealed, glass vials. All lenses were sterilized at 121° C. for 30 minutes in an autoclave prior to analysis.

### Example 9

Siloxane nano-particles formed via free-radical micro-emulsion polymerization of OHmPDMS and SiMAA DM in accordance with Examples 12-15 of US 2010/00249273 were added to HEMA in 50:50 weight ratio mixes. The resulting mixtures were found to have no miscibility. Dispersions within seconds of exposure to HEMA became unstable and immediately precipitated and/or aggregated upon visual observation. Without intending to be bound by theory, it is thought that the particles become unstable in the presence of HEMA because the surfactant, namely, DBS, is not bound to the dispersed silicone particles, and therefore, can interact and/or ultimately be dissolved by HEMA. Loss of stabilizing layer created by DBS on the surface of the silicone particles leads to exposure of hydrophobic particle surfaces, which interact with each other and lead to aggregation.

### Example 10

### Synthesis of PDMA macroCTA

Materials: N,N-dimethylacrylamide (DMA) was obtained from Jarchem and further purified via vacuum distillation. 4-cyano-4-[(dodecylsulfanylthiocarbonyl)sulfanyl]pentanoic acid was obtained from Sigma Aldrich and used as received. The photo-initiator, Irgacure 819, was obtained from Ciba Specialty Chemicals and used as received.

TABLE 15

| Macro- | CTA | DMA | CGI-819 | D3O | | |
|---|---|---|---|---|---|---|
| CTA | (g) | (g) | (g) | (g) | [M]:[CTA] | [CTA]:[I] |
| PDMA 25 | 2.000 | 12 | 0.403 | 0 | 24 | 5 |
| PDMA 50 | 0.977 | 12 | 0.203 | 0 | 50 | 5 |
| PDMA 75 | 1.090 | 20 | 0.056 | 20 | 75 | 20 |
| PDMA 100 | 0.815 | 20 | 0.084 | 20 | 100 | 10 |

Preparation of Polymerization Solution: The polymerization solution was prepared by adding an appropriate amount of distilled DMA and 3,7-dimethyl-3-octanol (D30) to an amber 60 mL glass jar. Next, the CTA and Irgacure-819 were added to the monomer and warmed/stirred to ensure homogeneity. The amber jar containing the final polymerization solution was sealed with a rubber septum and purged for 20 minutes with N₂ to remove O₂ from the solution. Finally the sealed jar was placed in an N₂ glove-box for storage.

Cure Conditions: The polymerization solution was cured under an N₂ atmosphere with 4 standard Phillips TL 20W/03 RS bulbs at intensity of 2.0 mW/cm². Prior to curing, the polymerization solution was poured into crystallization dish, which was then placed on a reflective glass surface underneath the TL-bulbs. The solution was irradiated for 1.5 hours and the resulting glassy polymer was dissolved in ethanol and precipitated from diethylether.

Purification of PDMA: After curing, the resulting polymerized material was dissolved in 40 mL of ethanol. The solution was stirred overnight then transferred to an addition funnel using 20 mL of ethanol to rinse out the crystallization dish. The polymer solution was added drop-wise to vigorously stirring diethyl ether to precipitate product. The precipitated polymer was dried in vacuo for several hours and then subjected to further purification via Soxhlet Extraction with diethyl ether. The polymer was analyzed for MW and MWD via SEC-MALLS.

### Example 11

### Synthesis of Poly(SA1)/PDMA Core/Shell Particles

To a 20 mL scintillation vial was added 1.0 g of poly(dimethylacrylamide) (PDMA) macroRAFT agent (macroCTA) from Example 11, containing a dodecyltrithiocarbonate end-group. The PDMA macroCTA was dissolved in 3 mL of DI water and the mixture was stirred magnetically for two hours. Once a homogeneous, yellow, viscous solution was obtained, 0.6 g of SA1 was added dropwise while stirring. The “milky” mixture was then sonicated for 1.5 hours at elevated temperatures (60-70° C.). The emulsified liquid was then placed under a nitrogen blanket and 5.6 mg of V-501 diazo-initiator ((Z)-4,4′-(diazene-1,2-diyl(bis(4-cyanopentanoic acid) (Wako USA) in 100 microL water was added to the emulsion. Prior to addition of the initiator solution, the V-501 was solubilized with 3-4 equivalents of NaHCO₃. The final mixture was polymerized for 2 hours at 60° C., after which time, the temperature was reduced to 25° C. The emulsion was stirred in all steps of the synthesis. The target degree of polymerization (DP) of SA1 at 100% conversion was fixed at 10 and macroCTA/Initiator ratio was maintained at 5:1. All mini-emulsion polymerization conditions are included in the table below in Table 16.

TABLE 16
Parameters and conditions used in the heterogeneous RAFT
polymerization of SA1 in the presence of a 10,000 g/mole PDMA
macroCTA

| DMA | | | |
|---|---|---|---|
| macroCTA | Mass of DMA | Moles of DMA | wt % |
| MW | macroCTA | macroCTA | Solids |
| 10000 g/mole | 1 g | 0.0001 | 34.75 |
| SA1 MW | Mass of SA1 | Moles of SA1 | |
| 598 g/mole | 0.6 g | 0.001 | |
| | Mass of V-501 | Moles of V-501 | |
| | 0.0056 g | 0.00002 | |
| | | | Target |
| CTA:I | Mass of Water | Target DP | MW |
| 5 | 3 g | 10 | 5980 |

Table 16 provides exemplary parameters and conditions for heterogeneous RAFT polymerization of SA1 in the presence of a 10,000 g/mole PDMA macroCTA. Other examples were prepared using macro-CTA of varying molecular weights under the same paramters and conditions. The final z-average particle diameter of the particles of the emulsions were measured via dynamic light scattering. [CS to provide MW data] Table 17 provides particle size diameter for each of the emulsions prepared by varying molecular weight macro-CTAs.

TABLE 17
Emsulsion particles sizes resulting from heterogeneous RAFT
polymerization of SA1 in the presence of PDMA macroCTAs of varying
molecular weights.

| | Mn | | |
|---|---|---|---|
| Macro- | g/mol | Mn | Particle Size diameter |
| CTA | (nominal) | g/mol | (nm) |
| PDMA | 5000 | 5100 | 179.6 |
| 50 | | | |
| PDMA | 7500 | 6900 | 197.3 |
| 75 | | | |
| PDMA | 10000 | 10720 | 256.0 |
| 100 | | | |

### Example 12

### Preparation of Particles Including Therapeutic Agent

A silicone monomer dispersion is prepared with 3 grams of polyethylene glycol azo macroinitiator, MW 4000 g/mol, in 9 grams water with a total of 3 grams of a mixture of SiMAA₂ DM and OHmPDMS. The ratio of SiMAA₂ DM: OHmPDMS is 45:55. To this mixture is added 0.5 grams cyclosporine. The mixture is emulsified into a mini-emulsion and allowed to polymerize to form a finished emulsion having an average particles size of less than 500 nm.

The finished emulsion is a viscous white fluid with no visible coagulum present. The emulsion is freely soluble in DI water and in HEMA.

### Example 13

Portions of the finished emulsion prepared in Example 5 were separately dispersed at weight ratios of 50:50 in N,N-dimethylacrylamide, N-vinylpyrrolidone, polyethylene glycol (400) monomethacrylate, and N-vinylformamide.

The resulting dispersions were freely soluble and stable in the monomers, and showed no signs of aggregation. Therefore, the reactive initiator-stabilized silicone microparticles of the present invention are dispersible in a wide variety of organic liquids, including the demonstrated neutral, hydrophilic vinyl monomers.

### Example 14

The finished emulsion of Example 5 was dispersed in 2-hydroxyethyl methacrylate such that the solids (reactive stabilizer and silicone polymer) to 2-hydroxyethyl methacrylate weight ratio was 60:40 by weight. The dispersion was poured into a drying tray, and was allowed to evaporate overnight under ambient conditions.

Not intending to be bound by theory, water was preferentially removed from the dispersion by the faster evaporation rate of water (relative to HEMA). Thus, the amount of solids and HEMA essentially remained constant, while the amount of water gradually decreased by evaporation. Furthermore, throughout the entire concentration process, the particles were constantly in the dispersed state in a liquid.

The resulting concentrated dispersion was a translucent white, waxy, semi-solid material that was approximately 60% by weight solids in 2-hydroxyethyl methacrylate. The concentrated dispersion was soluble and stable in HEMA.

### Example 15

### Comparative

Portions of the finished emulsion of Example 5 were separately dried overnight under ambient conditions or by lyophilization at zero degrees Celsius. The resulting dried white solids were dispersed in 2-hydroxyethyl methacrylate at a weight ratio of 40:60 (solids to HEMA). The resulting materials were translucent gels having large amounts of aggregation under optical microscopy. Thus, the particles of the present invention are not redispersible or stable after passing through a substantially dried (i.e., approaching 100 wt % concentrated) state.

### Example 16

### Comparative

A silicone monomer solution comprised of 4.5 g of SiMAA₂ DM and 5.5 g of OHmPDMS. To the monomer solution was added 0.1 g of a conventional oil-soluble initiator, 2,2′-azobismethylbutyronitrile (AMBN). A solution containing 3 g of polyethylene glycol (M.W. 4,000 g/mol) in 9 g of deionized water was prepared separately. To the polyethylene glycol solution was added 3 g of the silicone monomer solution. The resulting emulsion was then homogenized by sonication according to the procedures in Example 1 to give a miniemulsion. The miniemulsion was then polymerized according to the procedures in Example 1.

The resulting material contained a substantially clear liquid phase and a translucent solid polymer phase. The solid polymer was brittle, and could not be dispersed finely or dissolved in 2-hydroxyethyl methacrylate. The physical adsorption of the polyethylene glycol molecules on the droplet/particle surfaces was not sufficient to keep the particles stable. Thus, it is shown that the covalent binding of the polyethylene glycol molecules to the particle surface by the decomposition of the reactive macroinitiator (as in Examples 1 through 6) is essential for particle stability during polymerization, as well as for the dispersibility of the final particles in monomer.

### Example 17

### Comparative

To a silicone polymer microemulsion prepared from OHmPDMS and SiMAA₂ DM in accordance with Examples 12-15 of US 2010/00249273 was added 10% by weight of polyethylene glycol (M.W 4,000 g/mol). The dispersion was mixed overnight to ensure complete dissolution and adsorption of the PEG molecules on the particle surfaces. The resulting viscous, translucent dispersion was mixed with 2-hydroxyethyl methacrylate at a 50:50 weight ratio. The mixture immediately formed an opaque white liquid containing visible coagulum. Under optical microscopy, many large aggregates of particles were present, as in FIG. 9. Therefore, it is demonstrated that the post-addition of a PEG stabilizer to a silicone emulsion is not effective in keeping the particles dispersed in monomer if the PEG chains are only physically bound, rather than chemically bound, to the particle surfaces.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed is:

1. A contact lens formed from a composition comprising a plurality of engineered particles having an average particle size of less than about 500 nm dispersed in a monomer system, each of the engineered particles comprising a hydrophobic core and a hydrophilic shell,
wherein the hydrophobic core comprises a silicone-based polymer comprising multiple cross-links and the hydrophilic shell is formed from a reactive stabilizer, wherein a residue of the reactive stabilizer covalently bonds to the silicone-based polymer to form the particles; and
wherein the contact lens has a center thickness in the range of about 50 to about 180 micron and a haze that is less than 100% as compared to a CSI lens.

2. The contact lens of claim 1, wherein at least 50% by weight of the hydrophilic shell is the residue of the reactive stabilizer.

3. The contact lens of claim 2, wherein 100% by weight of the hydrophilic shell is the residue of the reactive stabilizer.

4. The contact lens of claim 1, wherein the shell is cross-linked.

5. The contact lens of claim 1, wherein the composition is substantially surfactant-free.

6. The contact lens of claim 1, wherein the residue of the reactive stabilizer comprises polyethylene glycol (PEG), poly(N,N-dimethylacrylamide) (PDMA) polyvinylpyrrolidone (PVP), poly(2-hydroxypropylmethacrylamide) (PHEMA), poly(N-2-hydroxypropylmethacrylamide) (PHPMA) poly(N,N-dimethylacrylamide-co-3-acrylamidopropanoic acid) (poly(DMA-co-ACA1.0), poly(N,N-dimethylacrylamide-co-4-acrylamidobutanoic acid) (poly(DMA-co-ACA1.5), poly(N,N-dimethylacrylamide-co-5-acrylamidopentanoic acid) (poly(DMA-co-ACA2.0), and combinations thereof.

7. The contact lens of claim 1, wherein the reactive stabilizer comprises a polyethylene glycol diazo polymer having a molecular weight in the range of about 1000 to about 10,000 g/mol.

8. The contact lens of claim 7, wherein the reactive stabilizer comprises a polyethylene glycol diazo polymer having a molecular weight in the range of about 2000 to about 6000 g/mol.

9. The contact lens of claim 8, wherein the reactive stabilizer comprises a polyethylene glycol diazo polymer having a molecular weight of about 4000 g/mol.

10. The contact lens of claim 1, wherein the reactive stabilizer comprises a polydimethylacrylamide thiocarbonate polymer having a molecular weight in the range of about 5000 to about 8000 g/mol.

11. The contact lens of claim 1, wherein the hydrophobic core comprises from about 0.1 to about 99.9% by weight of a siloxy macromer.

12. The contact lens of claim 11, wherein the hydrophobic core comprises from about 0.1 to about 50% by weight of the siloxy macromer.

13. The contact lens of claim 1, wherein the hydrophobic core comprises a siloxy macromer selected from the group consisting of methyl-bis(trimethylsilyloxy)-silyl-propylglycerol-methacrylate (SiMAA₂), mono-(3-methacryloxy-2-hydroxypropyloxy)propyl terminated, mono-butyl terminated polydimethylsiloxane), (OHmPDMS), monomethacryloxypropyl terminated mono-n-butyl terminated polydimethylsiloxane (mPDMS), N-(3-(3-(9-butyl-1,1,3,3,5,5,7,7,9,9-decamethylpentasiloxanyl) propoxy)-2-hydroxypropyl)acrylamide) (SA1), and SA2, as shown in the following formula: 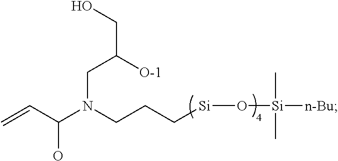
and combinations thereof.

14. The contact lens of claim 1, wherein the hydrophobic core comprises a siloxy macromer and the cross-links are formed in the absence of a cross-linker.

15. The contact lens of claim 1, wherein the cross-links are formed by a compound selected from the group consisting of Methyl-bis(trimethylsilyloxy)-silyl-propylglycerol-dimethacrylate (SiMAA₂ DM), monomethacryloxypropyl terminated mono-n-butyl terminated polydimethylsiloxane dimethacrylate (mPDMS DM), and combinations thereof.

16. The contact lens of claim 1, wherein the core further comprises a therapeutic agent.

17. The contact lens of claim 16, wherein the therapeutic agent is selected from the group consisting of immunosuppressant drugs, anti-microbial agents, antifungal agents, vitamins, anti-inflammatory agents, anti-VEGF (vascular epithelial growth factor) agents, macular pigment supplements, antibiotics, intraocular pressure reducing agents, and combinations thereof.

18. The contact lens of claim 16 where the therapeutic agent exhibits controlled release from by the core.

19. The contact lens of claim 1, wherein the core further comprises one or more modulating polymers such that the particles have a refractive index that is within about 10% of the refractive index of the hydrated contact lens.

20. The contact lens of claim 1, wherein the particles have a refractive index in the range of about 1.37 to about 1.47.

21. The contact lens of claim 1, wherein the contact lens has an oxygen permeability at least about 10 barrer more than a comparative contact lenses without the particles.

22. A composition comprising a plurality of engineered particles having an average particle size of less than about 500 nm dispersed in a monomer system, each of the engineered particles comprising a hydrophobic core and a hydrophilic shell,
wherein the core comprises a reaction product of at least one silicone reactive monomer and a hydrophobic segment of a reactive stabilizer comprising an amphiphilic macro-RAFT agent and the shell comprises one or more hydrophilic segments of the amphiphilic macro-RAFT agent.

23. The composition of claim 22, wherein the shell is cross-linked.

24. The composition of claim 22, wherein the hydrophilic segment of the reactive stabilizer comprises polyethylene glycol (PEG), poly(N,N-dimethylacrylamide) (PDMA) polyvinylpyrrolidone (PVP), poly(2-hydroxypropylmethacrylamide) (PHEMA), poly(N-2-hydroxypropylmethacrylamide) (PHPMA) poly(N,N-dimethylacrylamide-co-3-acrylamidopropanoic acid) (poly(DMA-co-ACA1.0), poly(N,N-dimethylacrylamide-co-4-acrylamidobutanoic acid) (poly(DMA-co-ACA1.5), poly(N,N-dimethylacrylamide-co-5-acrylamidopentanoic acid) (poly(DMA-co-ACA2.0), and combinations thereof.

25. The composition of claim 22, wherein the hydrophilic segment of the reactive stabilizer comprises a polydimethylacrylamide thiocarbonate polymer having a molecular weight in the range of about 5000 to about 8000 g/mol.

26. A method of preparing a plurality of engineered particles for dispersion in a monomer system, comprising:
providing a solution comprising a reactive stabilizer;
adding one or more siloxy monomers or macromers and optionally a cross-linker to the solution to form a mixture;
emulsifying the mixture to form a mini-emulsion;
polymerizing the mini-emulsion to form a polymeric dispersion that comprises a plurality of engineered particles each of which comprises a hydrophobic polymeric core and a hydrophilic shell, wherein the hydrophilic shell is formed from the reactive stabilizer.

27. The method of claim 26, wherein a residue of the reactive stabilizer covalently bonds to the silicone-based polymer to form the particles.

28. The method of claim 26, wherein one or more hydrophilic segments of the reactive stabilizer form the shell.

29. The method of claim 26, wherein the cross-linker is hydrophobic.

30. The method of claim 26, wherein the particles have an average particle size of less than about 500 nm.

31. The method of claim 26 further comprising increasing the concentration of the engineered particles in the polymeric dispersion by removing solution solvent to form a concentrated dispersion and subsequently adding the concentrated dispersion into the monomer system.

32. The contact lens of claim 1, wherein the contact lens has an oxygen permeability at least about 20 barrers more than a comparative contact lenses without the particles

33. The composition of claim 22, wherein the core is cross-linked.
